(19) United States
(12) Patent Application Publication
(10) Pub. No.: US 2015/0132410 A1
(43) Pub. Date: May 14, 2015

(54) COMPOSITIONS AND METHODS FOR THE PREVENTION AND TREATMENT OF ALCOHOL-INDUCED HANGOVER SYNDROME

(71) Applicant: Michael M. Jacobs, Pensacola, FL (US)

(72) Inventor: Michael M. Jacobs, Pensacola, FL (US)

(21) Appl. No.: 14/539,776

(22) Filed: Nov. 12, 2014

Related U.S. Application Data
(60) Provisional application No. 61/904,415, filed on Nov. 14, 2013.

(51) Int. Cl. A61K 31/192; A61K 31/341; A61K 31/495; A61K 31/426; A61K 33/06; A61K 31/525; A61K 31/4415; A61K 31/714; A61K 33/08; A61K 31/4545; A61K 31/51

(52) U.S. Cl. CPC A61K 31/192; A61K 31/4545; A61K 31/341; A61K 31/495; A61K 31/426; A61K 31/51; A61K 31/525; A61K 31/4415; A61K 31/714; A61K 33/08; A61K 33/06
USPC 424/692; 514/290; 514/255.04; 514/52; 514/276; 514/251; 514/345

(57) ABSTRACT

The invention provides compositions for the prevention and treatment of the symptoms of alcoholic beverage-induced hangover syndrome, related kits and methods of using the compositions and kits.

### CROSS-REFERENCE TO RELATED APPLICATIONS

This application claims the benefit of U.S. provisional application Ser. No. 61/904,415 filed Nov. 14, 2013 which is hereby incorporated by reference in its entirety.

### FIELD OF THE INVENTION

The invention relates to the field of orally administered hangover remedies.

### BACKGROUND OF THE INVENTION

Hangover is a complex phenomenon resulting from not only the ethyl alcohol (ethanol) component of alcoholic beverages but also from the milieu of organic and non-organic compounds found in particular alcoholic beverages, such as beer, wine, and liquors, and their metabolites. Hangover is best described as a syndrome with a wide spectrum of symptoms in response to highly variable amounts of alcohol consumption and individual (idiosyncratic) reactions. Typical symptoms of hangover include headache, malaise, gastrointestinal upset, nausea, and a generalized altered sense of well-being. Historically, many commonly available analgesic drugs as well as folklore remedies have been used in the treatment of hangovers resulting from the consumption of alcoholic beverages. However, no known approach has proven to be adequate.

What is needed and provided by the present invention are improved compositions and methods for preventing and treating hangover symptoms.

### SUMMARY OF THE INVENTION

One embodiment of the invention provides a composition for the prevention and/or treatment of alcoholic beverage-induced hangover that includes:

at least one, such as one, non-steroidal anti-inflammatory drug (NSAID), such as at least one prostaglandin synthesis inhibitor NSAID, such as at least one propionic acid derivative NSAID, for example naproxen sodium;

at least one, such as one, H1 antagonist anti-histamine, for example, loratadine or cetirizine;

at least one, such as one, H2 antagonist anti-histamine, for example, ranitidine or famotidine;

optionally one or more B vitamins selected from the group consisting of vitamins B1, B2, B6 and B12; and

optionally a pharmaceutically acceptable magnesium salt such as magnesium citrate, magnesium aspartate or magnesium hydroxide.

Also provided by the invention are related kits and methods for the prevention and/or treatment of alcoholic beverage-induced hangover syndrome.

Additional features, advantages, and embodiments of the invention may be set forth or apparent from consideration of the following detailed description, drawings, and claims. Moreover, it is to be understood that both the foregoing summary of the invention and the following detailed description are exemplary and intended to provide further explanation without limiting the scope of the invention as claimed.

### DETAILED DESCRIPTION OF THE INVENTION

The invention provides combination therapeutic compositions, kits including a combination of therapeutic compositions and methods utilizing a combination of therapeutic compounds for the prevention and treatment of the symptoms of hangover syndrome resulting from the consumption of alcoholic beverages.

One embodiment of the invention provides a composition for the prevention and/or treatment of alcoholic beverage-induced hangover that includes:

at least one, such as one, non-steroidal anti-inflammatory drug (NSAID), such as at least one prostaglandin synthesis inhibitor NSAID, such as at least one propionic acid derivative NSAID;

at least one, such as one, H1 antagonist anti-histamine; and

at least one, such as one, H2 antagonist anti-histamine.

The composition may also include one or more or all of vitamins B1, B2, B6 and B12. In one variation, the composition does not include vitamin B3 (niacin). In another variation, the composition does not include B vitamins other than vitamins B1, B2, B6 and B12.

The composition may also include one or more pharmacologically acceptable magnesium salts. Most preferably, the magnesium salt is a high bioavailability magnesium salt such as magnesium citrate, magnesium sulfate, magnesium aspartate or magnesium hydroxide.

Another embodiment of the invention provides a kit for the prevention and/or treatment of alcoholic beverage induced hangover that includes:

at least one discrete individual dosage grouping, such as one or a plurality of said groupings, of two or more separate component solid oral dosage forms, said forms collectively including/providing:

at least one, such as one, non-steroidal anti-inflammatory drug (NSAID), such as at least one prostaglandin synthesis inhibitor NSAID, such as at least one propionic acid derivative NSAID;

at least one, such as one, H1 antagonist anti-histamine; and

at least one, such as one, H2 antagonist anti-histamine.

The two or more solid oral dosage forms may also collectively include/provide one or more or all of vitamins B1, B2, B6 and B12. In one variation, the two or more solid oral dosage forms do not include vitamin B3 (niacin). In another variation, the two or more solid oral dosage forms do not include B vitamins other than vitamins B1, B2, B6 and B12.

The two or more solid oral dosage forms may also include/provide one or more pharmacologically acceptable magnesium salts. Most preferably, the magnesium salt is a high bioavailability magnesium salt such as magnesium citrate, magnesium sulfate, magnesium aspartate or magnesium hydroxide.

In such a kit, the various pharmaceutical, vitamin and mineral ingredients may be apportioned between separate solid oral dosage forms in any manner and combination. For example, each of the pharmaceuticals (alone or collectively), B vitamins (alone or collectively), and magnesium salt may be provided in separate solid oral dosage forms. The pharmaceutical ingredients may, for example, be provided in one solid oral dosage form. The B-vitamins (collectively) and the magnesium salt may, for example, be provided in one separate combined solid oral dosage form (separate and discrete from the dosage form containing the pharmaceutical ingredients) or in a separate solid oral dosage form for the B-vitamins (collectively) and a separate solid dosage form for the magnesium salt (which are separate and discrete from the dosage form containing the pharmaceutical ingredients).

A further embodiment of the invention provides a kit for the prevention and/or treatment of the symptoms of alcoholic beverage-induced hangover syndrome that includes:

at least one discrete individual dosage grouping of separate component solid oral dosage forms, including:
a first component which is a solid oral dosage form including non-steroidal anti-inflammatory drug (NSAID), such as at least one prostaglandin synthesis inhibitor NSAID, such as at least one propionic acid derivative NSAID; and
a second component which is a solid oral dosage form including at least one anti-histamine selected from the group consisting of an H1 antagonist and an H2 antagonist.

In one variation, the first component does not include an anti-histamine and the second component does not include a non-steroidal anti-inflammatory drug (NSAID).

In another variation, the second component comprises both an H1 antagonist and an H2 antagonist, and the first component does not include an antihistamine.

In another variation, the grouping further includes a third component, the third component including an H1 antagonist but not an H2 antagonist, the second component including an H2 antagonist but not an H1 antagonist and the first component not including an antihistamine.

In the embodiment and any of its variations, the grouping may further include a component including a magnesium salt, such as magnesium citrate, magnesium aspartate or magnesium hydroxide.

In the embodiment and any of its variations, the grouping may include a component that includes one or more or all of vitamins B1, B2, B6 and B12. In one variation, at least said component, e.g., the entire grouping of components, does not include vitamin B3 (niacin). In another variation, at least said component, e.g., the entire grouping of components, does not include B vitamins other than vitamins B1, B2, B6 and B12.

A further embodiment of the invention provides a kit for the prevention and/or treatment of alcoholic beverage induced hangover that includes:

at least one discrete individual dosage grouping of separate component solid oral dosage forms, including:

a first component which is a solid oral dosage form that includes:
a non-steroidal anti-inflammatory drug (NSAID), such as at least one prostaglandin synthesis inhibitor NSAID, such as at least one propionic acid derivative NSAID,
an H1 antagonist anti-histamine, and
an H2 antagonist anti-histamine;

a second component which is a solid oral dosage form that includes one or more or all of vitamins B1, B2, B6 and B12.

The second component may, for example, not include any of an NSAID, H1 antagonist and H2 antagonist and first component may, for example, not include any of vitamins B1, B2, B6 and B12 or any or any substantial amount of any B vitamin at all. In one variation, at least the second component, e.g., the entire grouping of components, does not include vitamin B3 (niacin). In another variation, the first and second components, e.g., the entire grouping of components, does not include B vitamins other than vitamins B1, B2, B6 and B12.

The first or second component may further include a magnesium salt such as magnesium citrate, magnesium sulfate, magnesium aspartate or magnesium hydroxide. The grouping may include a third component that includes include a magnesium salt such as magnesium citrate, magnesium aspartate or magnesium hydroxide and the first and second component do not include a magnesium salt.

In the kits of the invention, the individual dosage groupings may be provided in any manner, such as any manner known in the art. The kits of the invention may, for example, include a plurality of individual dosage groupings consisting of a plurality of individually packaged and sealed plastic/polymer bags containing the dosage forms of the individual dosage grouping. Alternatively (or in addition), the kits of the invention may include a plurality of individual dosage groupings presented in one or more blister packs. Each blister pack may, for example, include one individual dosage grouping of components. Alternatively, a single blister pack may include more than one individual dosage grouping of components, for example, each dosage grouping being geographically (spatially) segregated on the blister pack. In the case in which a single blister pack includes more than one geographically segregated individual dosage groupings, the blister pack may optionally be perforated to permit detachment of segments containing an individual dosage grouping from the main blister pack, as known in the art.

Still another embodiment of the invention provides a method for the prevention and/or treatment of alcoholic beverage-induced hangover that includes the step of: orally administering, such as self orally administering, at one time (co-administering):

at least one, such as one, non-steroidal anti-inflammatory drug (NSAID), such as at least one prostaglandin synthesis inhibitor NSAID, such as at least one propionic acid derivative NSAID;
at least one, such as one, H1 antagonist anti-histamine; and
at least one, such as one, H2 antagonist anti-histamine.

Said oral administration step may also include oral administration of one or more or all of vitamins B1, B2, B6 and B12. In one variation, said administration does not include administering vitamin B3 (niacin). In another variation, said administration does not include administering B vitamins other than vitamins B1, B2, B6 and B12.

Said oral administration step may also include oral administration of one or more magnesium salts such as magnesium citrate, magnesium sulfate, magnesium aspartate or magnesium hydroxide.

For prevention of hangover, the oral administration step may, for example, be performed in advance of consuming (imbibing) alcoholic beverages, for example, 30 minutes to 120 minutes, such as 60 minutes, before commencing consumption of alcoholic beverages. Oral administration may, for example, be repeated again 8-12 hours later, whether or not hangover symptoms have developed. For prevention of hangover, the oral administration may in addition or alternatively be performed after consumption of alcoholic beverages has commenced but before the onset of hangover symptoms. To treat hangover symptoms, initial administration may occur upon initiation of the symptoms or any time after their onset and then optionally repeated for example at intervals of 4-8 hours.

The method may, for example, include orally administering the composition embodiments and variations thereof described herein. The method may, for example, include orally administering an individual dosage grouping of components from a kit embodiment described herein. By administering the enumerated/delineated elements at one time or co-administering the elements, what is meant is that they are taken together over a short period of time but not necessarily in the same instant. Thus, for example, in pill form, separate pills containing the elements may be ingested in a sequential manner over the course of a short period of time such as within five minutes.

It should be understood that by prevention of hangover what is meant is at least partial prevention of the development of hangover or some mitigation of symptoms of hangover syndrome, if they should occur, and not necessarily complete prevention of hangover. Similarly, it should be understood that by treatment of hangover what is meant is at least partial relief from (or at least partial mitigation of) the symptoms or some of the symptoms of hangover syndrome and not necessarily complete resolution of the symptoms of hangover syndrome.

Particular examples of compounds, compound combinations and their dosing for use in embodiments of the invention are provided as follows.

Most NSAIDs act as nonselective inhibitors of the enzyme cyclooxygenase (COX), inhibiting the cyclooxygenase-1 (COX-1) and cyclooxygenase-2 (COX-2) isoenzymes. This inhibition is competitively reversible (with varying degrees of reversibility), as opposed to the mechanism of aspirin, which is irreversible inhibition. COX catalyzes the formation of prostaglandins and thromboxane from arachidonic acid. Prostaglandins act as, among other things, messenger molecules in the process of inflammation. More recently, NSAIDs that selectively inhibit COX-2, such as celecoxib, have been developed.

Among NSAIDs that may be used in the various embodiments of the present invention are the propionic derivative class of progesterone synthesis-inhibiting NSAIDs including the following compounds or pharmaceutically acceptable salts thereof (individual adult dosing examples shown parenthetically):

Naproxen (naproxen sodium: 50-300 mg, e.g., 220 mg);

Ibuprofen (50-400 mg, e.g. 100-400 mg, e.g. 100-200 mg);

Dexibuprofen (100-450 mg);

Fenoprofen (fenoprofen calcium: 100-600 mg, e.g., 300-600 mg);

Ketoprofen (25-150 mg, e.g., 50-75 mg);

Dexketoprofen (10-30 mg);

Flurbiprofen (20-100 mg, e.g. 50 mg);

Oxaprozin (10-300 mg); and

Loxoprofen (20-120 mg, e.g., 60-120 mg).

H1 antagonists (H1RA) are antagonists of the histamine H1 receptor that reduce or eliminate effects mediated by histamine, an endogenous chemical mediator released during allergic reactions, some of which can be induced by individual susceptibilities to various ingredients of beers, wines and liquors.

Second and third generation H1 antagonists are preferred for use in the invention as they are more selective than first generation H1 antagonists for peripheral H1 receptors as opposed to central nervous system H1 receptors and cholinergic receptors. This selectivity significantly reduces the occurrence of adverse drug reactions, such as sedation, while still providing effective relief of peripheral histamine-related conditions. Second generation H1 antagonists that may be used in embodiments of the present invention include the following compounds or pharmaceutically acceptable salts thereof (individual adult dosing examples shown parenthetically):

Ketotifen (0.5-1.0 mg, e.g. 1.0 mg);

Cetirizine (cetirizine hydrochloride (HCL): 5-20 mg, e.g. 10 mg);

Loratadine (5-20 mg, e.g. 10 mg);

Rupatadine (5-20 mg, e.g. 10 mg);

Mizolastine (5-20 mg, e.g. 10 mg);

Acrivastine (5-20 mg, e.g. 8-10 mg);

Ebastine (5-20 mg, e.g. 10 mg);

Bilastine (10-40 mg, e.g. 20 mg); and

Quifenadine (quifenadine HCL: 10-50 mg, e.g. 25 mg).

Third-generation H1-antihistamines are the active enantiomer (levocetirizine) or metabolite (desloratadine & fexofenadine) derivatives of second-generation drugs intended to have increased efficacy with fewer adverse drug reactions. Third-generation H1 antagonists that may be used in embodiments of the present invention include the following compounds or pharmaceutically acceptable salts thereof (individual adult dosing examples shown parenthetically):

Levocetirizine (levocetirizine dihydrochloride: 0.5-5.0 mg, e.g. 1.0 mg);

Desloratadine (1.0-10 mg, e.g. 5.0 mg); and

Fexofenadine (fexofenadine HCL: 10-80 mg, e.g. 50 mg).

Histamine H2 receptor antagonists (H2RA) block the action of histamine parietal cell H2 receptors in the stomach, thereby decreasing their production of acid. The H2 antagonist cimetidine in particular interferes with some of the body's mechanisms of drug metabolism and elimination through the liver cytochrome P450 (CYP) pathway, interfering with the body's normal metabolism of various drugs including ethanol. Accordingly, cimetidine is disfavored for use as an H2 antagonist in embodiments of the invention. Preferred H2 antagonists for use in embodiments of the invention include ranitidine, which is less potent than cimetidine in inhibiting the CYP system, and famotidine, which has negligible effect on the CYP system and no significant interactions. H2 antagonists that may be used in embodiments of the present invention include the following compounds or pharmaceutically acceptable salts thereof (individual adult dosing examples shown parenthetically):

Ranitidine (ranitidine HCL: 25-250 mg, e.g. 50-150 mg, e.g. 150 mg); and

Famotidine (10-50 mg, e.g. 20-40 mg, e.g. 20 mg).

Generally, the particular active pharmaceutical ingredients used in the various embodiments of the invention may be provided and/or administered in therapeutically effective amounts as set forth herein and/or as known in the art.

As described above, a complex of B vitamins including vitamins B1, B2, B6 and B12 may be used in the embodiments of the invention. These B vitamins are depleted as a result of consuming alcohol. The dose of each B vitamin used may, for example, be in the range of 1 to 150-times, 1 to 100-times, 1 to 50-times, 1 to 40-times, 1 to 30-times, 1 to 20-times, 10 to 150-times, 10 to 100-times, 10 to 50-times, 10 to 40-times, 10 to 30-times, or 10 to 20-times the daily recommended dietary allowance (RDA) for the vitamin, such as at or around 10 or 20-times the RDA of each B vitamin used. The RDA for each vitamin, as defined herein, is that recommended by the National Academy of Sciences, Institute of Medicine, Food and Nutrition Board for 19-30 year old males, specifically: vitamin B1 (thiamine) 1.2 mg/day; vitamin B2 (riboflavin) 1.3 mg/day; vitamin B6 (pyridoxine) 1.3 mg/day; and vitamin B12 (cyanocobalamine) 2.4 micrograms/day. The dosages of each B vitamin used may, for example, be as follows: vitamin B1 (thiamine) 100 mg; vitamin B2 (riboflavin) 100 mg; vitamin B6 (pyridoxine) 100 mg; and vitamin B12 (cyanocobalamine) 100 micrograms.

Magnesium in embodiments of the invention acts as a stomach buffer and is an essential co-factor in a number of enzymatic processes that impact hangover syndrome. Magnesium is depleted as a result of consuming alcohol. Magnesium salts used with embodiments of the invention may, for example, provide a dose of elemental magnesium in the range of 50-500 mg, such as 100-300 mg, such as 200 mg. Preferred magnesium salts for use with embodiments of the invention are magnesium citrate and magnesium aspartate due to their high dissolution and consequently enhanced absorption of elemental magnesium. However, other magnesium salts may also be used.

In general, the individual compounds/active ingredients may be present in and administered in amounts that are therapeutically effective in their combination for the prevention and/or treatment of hangover syndrome. Wherever in this disclosure it is described that a component or dosage form does not include a particular substance, what is meant is that there is none of the substance at all or that the amount of the substance therein does not exceed a trace amount. In addition, wherever in this disclosure it is described that a component or dosage form does not include a particular substance, the invention also provides corresponding embodiments in which the amount of the substance present therein is no more than 10%, 5% or 1% of the total amount of the substance across all components/dosage forms. Magnesium salts that may be used in embodiments of the invention include, for example, Magnesium aspartate, Magnesium citrate, Magnesium chloride, Magnesium sulfate, Magnesium lactate and Magnesium hydroxide.

Table 1 illustrates a preferred combination of active ingredients for use in embodiments of the invention, exemplifying an individual dosage, for the prevention and/or treatment of the symptoms of hangover syndrome resulting from consumption of alcoholic beverages. The elements of the combination may be provided in any manner such as in the composition embodiments described herein, or as the individual dosage groupings of the kit embodiments of the invention, or administered orally in one or more dosage forms as in the method embodiments of the invention.

TABLE 1

| Ingredient | Dosage |
|---|---|
| Naproxen sodium | 220 mg |
| Ranitidine | 150 mg |
| Loratadine | 10 mg |
| Vitamins B1, B2, B6 and B12 | 10-times RDA each |
| Magnesium citrate | 200 mg elemental |
| (or Magnesium aspartate, Magnesium hydroxide | Magnesium |
| or other Magnesium salt) | |

A related combination of active ingredients for use in embodiments of the invention may replace loratadine 10 mg with cetirizine 5 mg, with the other elements of the combination shown in Table 1 remaining the same. Similarly, ranitidine 150 mg in the combinations may be replaced by famotidine 20 mg. Thus, Table 2 below illustrates another preferred combination of active ingredients for use in embodiments of the invention, with single-use individual dosage amounts exemplified.

TABLE 2

| Ingredient | Dosage |
|---|---|
| Naproxen sodium | 220 mg |
| Famotidine | 20 mg |
| Ceterizine | 5 mg |
| Vitamins B1, B2, B6 and B12 | 10-times RDA each |
| Magnesium hydroxide | 200 mg elemental |
| (or Magnesium aspartate, Magnesium citrate | Magnesium |
| or other Magnesium salt) | |

Any of the compositions of the invention, or individual composition components of the kits of the invention, may optionally further include one or more pharmaceutically acceptable excipients. The various active pharmaceutical ingredients used in the invention are well known as is their formulation and use of excipients therewith. The compositions of the invention may be provided in an oral dosage form, such as solid oral dosage forms and/or liquid oral dosage forms, for example, in a syrup form. Solid oral dosage forms include without limitation tablets, capsules, and gel caps.

The compositions of the invention may also be provided in a water-dissolvable and/or—suspendable powder or friable form for mixing with water or a beverage before ingestion/consumption. Such powder or friable forms may, for example, be provided in a bulk form in a container from which individual dosage amounts can be withdrawn for use or as one or more individual sachets or packets each containing a single dosage of the composition. One embodiment of the invention provides a kit that includes a first sachet/packet that includes the pharmaceutical active ingredients (as described herein) and a second sachet/packet that includes one or more of such as all of B1, B2, B6 and B12. In a variation, the first or second sachet/packet further includes a pharmaceutically acceptable magnesium salt. All of the sachets/packet may exclude vitamin B3. In any of the compositions, components and methods of the invention in which vitamin B3 is not included, what is meant is that there is no vitamin B3 whatsoever or that the amount of vitamin B3 present does not exceed a trace amount.

In general, whether the various pharmaceutical or vitamin components of the invention are synthetic or naturally derived, it should be understood that they are provided in and used in at least substantially pure forms with or without excipients and mixed or unmixed with one or more of the other active ingredients according to the invention as described herein. Thus, the compositions and components of the invention do not include foods and are not foods. However, powder/friable forms of the compositions and components of the invention may, for example, be mixed with food derived beverages, such as fruit juices, for oral administration by drinking the mixture.

A solid oral dosage form may, for example, be a compressed dosage form, such as a compressed tablet or caplet, obtained by compressing a powder mixture. The powder mixture may contain one or more active ingredients and optionally one or more excipients, such as binders, disintegrants, lubricants, fillers and the like, as known in the art.

Suitable fillers for solid compressed oral dosage forms include, but are not limited to, water-soluble compressible carbohydrates such as sugars, which include dextrose, sucrose, isomaltalose, fructose, maltose, and lactose, polydextrose, sugar-alcohols, which include mannitol, sorbitol, isomalt, maltitol, xylitol, erythritol, starch hydrolysates, which include dextrins, and maltodextrins, and the like, water insoluble plastically deforming materials such as microcrystalline cellulose or other cellulosic derivatives, water-insoluble brittle fracture materials such as dicalcium phosphate, tricalcium phosphate and the like and mixtures thereof.

Suitable binders for solid compressed oral dosage forms include, but are not limited to, dry binders such as polyvinyl pyrrolidone, hydroxypropylmethylcellulose, and the like; wet binders such as water-soluble polymers, including hydrocolloids such as alginates, agar, guar gum, locust bean, carrageenan, tara, gum arabic, tragacanth, pectin, xanthan, gellan, maltodextrin, galactomannan, pusstulan, pullulan, laminarin, scleroglucan, gum arabic, inulin, pectin, whelan, rhamsan, zooglan, methylan, chitin, cyclodextrin, chitosan, polyvinyl pyrrolidone, cellulosics, starches, and the like; and derivatives and mixtures thereof.

Suitable disintegrants for solid compressed oral dosage forms include, but are not limited to, sodium starch glycolate, cross-linked polyvinylpyrrolidone, cross-linked carboxymethylcellulose, starches, microcrystalline cellulose, and the like.

Suitable lubricants for solid compressed oral dosage forms include, but are not limited to, long chain fatty acids and their salts, such as magnesium stearate and stearic acid, talc, and waxes.

Suitable glidants for solid compressed oral dosage forms include, but are not limited to, colloidal silicon dioxide, and the like.

Although the invention has been described in connection with specific preferred embodiments, it should be understood that the invention as claimed should not be unduly limited to these specific embodiments.

What is claimed is:

1. A pharmaceutical composition for the prevention and treatment of alcoholic beverage-induced hangover syndrome, comprising:
at least one prostaglandin synthesis inhibitor NSAID;
at least one H1 antagonist anti-histamine; and
at least one H2 antagonist anti-histamine.

2. The pharmaceutical composition of claim 1, further comprising one or more of vitamins B1, B2, B6 and B12.

3. The pharmaceutical composition of claim 1, further comprising vitamins B1, B2, B6 and B12.

4. The pharmaceutical composition of claim 1, further comprising a pharmaceutically acceptable magnesium salt.

5. The pharmaceutical composition of claim 2, further comprising a pharmaceutically acceptable magnesium salt.

6. The pharmaceutical composition of claim 3, further comprising a pharmaceutically acceptable magnesium salt.

7. The pharmaceutical composition of claim 1, wherein:
the at least one NSAID comprises a compound selected from the group consisting of naproxen, ibuprofen and pharmaceutically acceptable salts thereof;
the at least one H1 antagonist comprises a compound selected from the group consisting of loratidine, cetirizine and pharmaceutically acceptable salts thereof; and
the at least one H2 antagonist comprises a compound selected from the group consisting of ranitidine, famotidine and pharmaceutically acceptable salts thereof.

8. The pharmaceutical composition of claim 1, wherein the composition is in an oral dosage form.

9. The pharmaceutical composition of claim 8, wherein the oral dosage form is solid.

10. The pharmaceutical composition of claim 8, wherein the oral dosage form is liquid.

11. A kit for the prevention or treatment of alcoholic beverage induced hangover, comprising:
at least one discrete individual dosage grouping of two or more separate component solid oral dosage forms, said forms collectively providing:
at least one prostaglandin synthesis inhibitor NSAID;
at least one H1 antagonist anti-histamine; and
at least one H2 antagonist anti-histamine.

12. The kit of claim 11, wherein said forms collectively further provide one or more of vitamins B1, B2, B6 and B12.

13. The kit of claim 12, wherein said forms collectively provide vitamins B1, B2, B6 and B12.

14. The kit of claim 11, wherein said forms collectively further provide a pharmaceutically acceptable magnesium salt.

15. The kit of claim 12, wherein said forms collectively further provide a pharmaceutically acceptable magnesium salt.

16. The kit of claim 13, wherein said forms collectively further provide a pharmaceutically acceptable magnesium salt.

17. The kit of claim 12, comprising:
a first solid dosage form that comprises:
the at least one prostaglandin synthesis inhibitor NSAID,
the at least one H1 antagonist anti-histamine,
the at least one H2 antagonist anti-histamine,
wherein the first solid dosage form does not comprise vitamins B1, B2, B6 and B12; and
a second solid dosage form that comprises:
the one or more of vitamins B1, B2, B6 and B12,
wherein the second solid dosage form does not comprise any of an NSAID, an H1 antagonist anti-histamine and an H2 antagonist anti-histamine.

18. The kit of claim 14, comprising:
a first solid dosage form that comprises:
the at least one prostaglandin synthesis inhibitor NSAID,
the at least one H1 antagonist anti-histamine,
the at least one H2 antagonist anti-histamine,
wherein the first solid dosage form does not comprise the pharmaceutically acceptable magnesium salt; and
a second solid dosage form that comprises:
the pharmaceutically acceptable magnesium salt,
wherein the second solid dosage form does not comprise any of an NSAID, an H1 antagonist anti-histamine and an H2 antagonist anti-histamine.

19. A method for at least partially preventing the symptoms of hangover syndrome associated with the consumption of ethyl alcohol containing beverages by a human subject, comprising:
in advance of the subject consuming one or more ethyl alcohol containing beverages or after the subject has commenced consuming one or more ethyl alcohol containing beverages but before the subject has developed symptoms of hangover syndrome, orally co-administering to the subject:
at least one prostaglandin synthesis inhibitor NSAID,
at least one H1 antagonist anti-histamine, and
at least one H2 antagonist anti-histamine.

20. The method of claim 19, wherein the co-administering step further comprises:
orally co-administering one or more of vitamins B1, B2, B6 and B12.

21. The method of claim 20, wherein the co-administering step comprises:
orally co-administering vitamins B1, B2, B6 and B12.

22. The method of claim 19, wherein the co-administering step further comprises:
orally co-administering a pharmaceutically acceptable magnesium salt.

23. The method of claim 20, wherein the co-administering step further comprises:
orally co-administering a pharmaceutically acceptable magnesium salt.

24. The method of claim 21, wherein the co-administering step further comprises:
orally co-administering a pharmaceutically acceptable magnesium salt.

25. The method of claim 19, wherein the at least one prostaglandin synthesis inhibitor NSAID, the at least one H1 antagonist anti-histamine, and the at least one H2 antagonist anti-histamine are provided together in a single solid oral dosage form and the co-administering step comprises orally administering said single solid oral dosage form.

26. A method for treating hangover syndrome associated with the consumption of ethyl alcohol containing beverages, comprising:
to a human subject in need of treatment for an alcoholic beverage-induced hangover, orally co-administering:
at least one prostaglandin synthesis inhibitor NSAID,
at least one H1 antagonist anti-histamine, and
at least one H2 antagonist anti-histamine.

27. The method of claim 26, wherein the co-administering step further comprises:
orally co-administering one or more of vitamins B1, B2, B6 and B12.

28. The method of claim 27, wherein the co-administering step comprises:
orally co-administering vitamins B1, B2, B6 and B12.

29. The method of claim 26, wherein the co-administering step further comprises:
orally co-administering a pharmaceutically acceptable magnesium salt.

30. The method of claim 27, wherein the co-administering step further comprises:
orally co-administering a pharmaceutically acceptable magnesium salt.

31. The method of claim 28, wherein the co-administering step further comprises:
orally co-administering a pharmaceutically acceptable magnesium salt.

32. The method of claim 26, wherein the at least one prostaglandin synthesis inhibitor NSAID, the at least one H1 antagonist anti-histamine, and the at least one H2 antagonist anti-histamine are provided together in a single solid oral dosage form and the co-administering step comprises orally administering said single solid oral dosage form.
